(19) United States
(12) Patent Application Publication
(10) Pub. No.: US 2008/0281408 A1
(43) Pub. Date: Nov. 13, 2008

(54) Artificial Blood Vessel

(75) Inventors: Makoto Kodama, Fukuoka (JP); Takashi Kitajima, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: KYUSHU INSTITUTE OF TECHNOLOGY, Kitakyushu-shi (JP)

(21) Appl. No.: 11/574,538

(22) PCT Filed: Aug. 29, 2005
(86) PCT No.: PCT/JP05/15622

§ 371 Date: Oct. 22, 2007

(30) Foreign Application Priority Data
Sep. 3, 2004 (JP) 2004-257768

(51) Int. Cl. A61F 2/82

(52) U.S. Cl. 623/1.42

(57) ABSTRACT

It is an object of the present invention to retain on an artificial blood vessel material an endothelial cell growth-promoting agent, for example the angiogenic factor HGF, without impairing its activity, thereby providing an artificial blood vessel having the function of promoting endothelialization. Such an object can be attained by an artificial blood vessel that includes a porous tubular structure formed from, for example, polytetrafluoroethylene and, layered and immobilized in sequence onto at least the inner surface thereof, (1) a polyamino acid urethane copolymer, (2) collagen or gelatin, and (3) an endothelial cell growth-promoting agent having collagen-binding activity. Preferred examples of the endothelial cell growth-promoting agent include a fusion protein of a polypeptide having collagen-binding activity such as, for example, a fibronectin-derived polypeptide and an angiogenic factor, in particular, HGF.

### TECHNICAL FIELD

The present invention relates to an artificial blood vessel having endothelial cell growth-promoting activity and excellent biocompatibility.

### BACKGROUND ART

With regard to artificial blood vessels for replacing dysfunctional blood vessels that have become occluded or damaged, medium- or large-diameter artificial blood vessels have already been put into practical use, but the practical application of small-diameter artificial blood vessels having an internal diameter of 4 mm or less has been delayed because occlusion is easily caused by thrombus deposition after grafting. Artificial blood vessels that can be applied as a substitute for small-diameter blood vessels such as a coronary artery or a lower limb blood vessel (below-knee artery) have not yet been put into practical use, and in the current situation a living blood vessel from another site is grafted instead. However, patients having a disorder in such an artery often have fragile blood vessels in other sites, and it is difficult to obtain a blood vessel that can be used for substitution. Furthermore, it is difficult to apply angioplasty (stenting, balloon catheter treatment, etc.) to fragile blood vessels. There is therefore a strong desire for the practical application of small-diameter artificial blood vessels.

One of the initial phenomena that causes the occlusion of small-diameter artificial blood vessels is thrombus deposition. In the small-diameter blood vessel, the blood flow volume is low, and once thrombi have been deposited they do not come off but rather they advance the formation of thrombi to thus increase the thickness and result in occlusion. Because of this, there has been a demand for an antithrombogenic polymer material that is completely free from the attachment of plasma protein and blood platelets. Up until now, expanded polytetrafluoroethylene (ePTFE) has been considered to be the best material in terms of antithrombogenicity, and the use thereof for small-diameter blood vessels has been examined; although ePTFE suppresses the initial deposition of thrombi, after a long period of time thrombi are deposited and inevitably cause occlusion. It is also said that in practical use even medium- or large-diameter ePTFE artificial blood vessels need to be replaced every 2 years after grafting because of thrombus deposition.

Therefore, further searching for and improvement of antithrombogenic materials have been carried out, but only a few have been examined for an effect in grafting in a living body, and it is said that they are not yet adequate in terms of long-term antithrombogenicity. For example, Uchida et al. have reported a novel copolymer of polyurethane and polyamino acid (PAU) (ref. Non-Patent Publications 1 and 2). The polyamino acid site is hydrophilic and has affinity for cells, and the polyurethane site exhibits antithrombogenicity. JP-A-2001-136960 (Patent Publication 1) (JP-A denotes a Japanese unexamined patent application publication) discloses that a material coated with the above polymer suppressed the deposition of blood platelets for a long period of time and exhibited excellent antithrombogenicity. Furthermore, the endothelialization effect of a material coated with the above polymer has also been reported (ref. Non-Patent Publication 3). However, this result was obtained as a result of replacing a short length of about 5 mm of a blood vessel site of a rat abdominal aorta, and it is surmised that this result is due to the high repairability possessed by rats. In a graft experiment using a dog, endothelialization was not observed. Various other examples of antithrombogenic materials have also been disclosed, but their evaluation is often carried out in vitro, and none thereof show a sufficient level of effect in living bodies. An artificial blood vessel, etc. treated with a hydrophilic polymer, for example, a hydrophilic polyurethane, has been proposed (ref. Patent Publication 2), but its effect in a living body is not sufficient.
(Patent Publication 1) JP-A-2001-136960
(Patent Publication 2) Published Japanese translation No. 11-502734 of a PCT application
(Non-Patent Publication 1) J. Polym Sci. A: Polym. Chem. 37, 383-389; 1999
(Non-Patent Publication 2) Polymer 41, 473-480; 2000
(Non-Patent Publication 3) Wang et al., J. Biomed. Mater. Res. 62, 315-322; 2002

Because of these points, there is a limit to the improvement of the artificial blood vessel material itself, and as an alternative thereto there have been attempts to utilize the antithrombogenicity of endothelial cells. In blood vessels of a living body, thrombi are deposited on a site from which endothelial cells are peeled off, but a site in which endothelial cells are present is normally free from thrombus deposition. That is, it is surmised that it is an endothelial cell layer (endothelial lining) that actually carries an antithrombogenic function in living blood vessels, and the ultimate antithrombogenic material is vascular endothelial cells. A method based on this idea involves forming an endothelial lining by seeding endothelial cells in advance on an inner face of an artificial blood vessel substrate, and then grafting the artificial blood vessel to a human body. However, since this method requires steps of harvesting, culturing, etc. of cells, it cannot be used immediately. Furthermore, there is the undesirable point that harvesting cells imposes a burden on a patient.

Another method involves forming a composite of an artificial blood vessel material with a substance that induces endothelial cells to cover the surface of the blood vessel during an early stage after grafting the artificial blood vessel to a human body; an adhesive peptide or a matrix protein such as collagen or fibronectin (Fn) for promoting the adhesion of endothelial cells and a protein such as a growth factor for promoting the growth of endothelial cells have been examined. As a way to enhance cell adhesion, a method has been proposed in which a peptide sequence (RGD) involved in cell adhesion is immobilized on polyurethane by a covalent bond (ref. Non-Patent Publications 4, 5, and 6). Since the RGD sequence itself has no effect in growing cells, it is desirable that a mechanism for growing endothelial cells is provided.
(Non-Patent Publication 4) Lin et al., J. Biomater. Sci. Polymer Edn, 3, 217-227;
(Non-Patent Publication 5) Lin et al., J. Biomed. Mater. Res 28, 329-342; 1994
(Non-Patent Publication 6) Tiwari et al., FASB J. 16, 791-796; 2002

Immobilizing an extracellular matrix component (collagen, fibronectin, laminin, proteoglycan, etc.) is also considered to be effective for enhancing cell adhesion. For example, Vohra et al. have reported that it is possible to make fibronectin (Fn) adsorb on ePTFE (ref. Non-Patent Publication 7). In this case, immobilizing means such as covalent bonding is not employed, and although the amount thereof adsorbed is about 0.3 μg per cm², cells can be bound. Furthermore, about 70% of adsorbed Fn is still retained after passing phosphate buffered saline (PBS) at a flow rate of 200 mL per minute for 2 hours. However, for a practical artificial blood vessel, it is thought that a period of 2 hours is too short. Furthermore, it is not clear whether endothelial cells that have become bound to the Fn adsorption surface can remain bound under this flow rate, and it is surmised that adsorption alone is not sufficient. Moreover, it is hard to imagine that these results might be applied to in vivo environment since, unlike a culture experiment, a large quantity of endothelial cells (or precursor cells thereof) cannot be present in vivo.
(Non-Patent Publication 7) Artif. Organ 14, 41-45; 1990

As a method for more reliably immobilizing a protein, a method in which crosslinking immobilization is carried out using glutaraldehyde has often been carried out (ref. e.g. Patent Publication 3), but since the protein is denatured, this method is not desirable for a protein having activity, and when collagen is immobilized calcification occurs. A large number of methods in which immobilization is carried out by covalent bonding without using a protein-denaturing agent have been disclosed. For example, Hamaguchi et al. (ref. Non-Patent Publication 8) have carried out graft polymerization of an ePTFE tube with methacrylic acid by treating the tube with an alkali metal compound (methyllithium) to thus abstract a fluorine atom. Gelatinized collagen was immobilized here by covalent bonding using carbodiimide. It is stated that the artificial blood vessel thus formed had no influence on the patency rate but the percentage endothelialization of the surface by endothelium after 4 weeks was superior. However, ePTFE on which gelatin had not been immobilized was also endothelialized in the same way after 12 weeks. Nishibe et al. has reported that fibronectin is covalently bonded in the same manner and in this case the coverage with endothelium is improved (ref. Non-Patent Publication 9).
(Patent Publication 3) JP-A-8-283667
(Non-Patent Publication 8) Jinkouzouki (Artificial Organs) 24, 168-173; 1995
(Non-Patent Publication 9) Surg. Today, 30, 426-431; 2000

In summary, these methods are methods in which a material is chemically or physically modified and methacrylic acid, etc. is bonded to the modified site by graft-polymerization, etc., thus forming on the surface a strongly reactive functional group (a hydroxy group, an epoxy group, a carboxyl group, etc.). Subsequently, this functional group is directly contacted with a protein molecule or covalently bonded thereto using a crosslinking agent; a large number of patents relating to artificial blood vessels based on this method have been published (ref. e.g. Patent Publications 4, 5, and 6). As a material that is to be immobilized, there are matrix proteins such as Fn and collagen, TGF α, insulin, a growth factor such as fibroblast growth factor (FGF), etc. and, furthermore, immobilizing heparin as an antithrombotic agent has been proposed. Transferrin can also be used. It is also proposed to immobilize them in combination. In these methods, unlike the case in which glutaraldehyde is used, the protein is not denatured, but a protein cannot be covalently bonded without any structural change. This might affect the activity of the protein. It should be noted that the above-mentioned series of patents disclose that various types of protein can be bonded, but they do not disclose a method for immobilizing any protein or growth factor without affecting its activity.
(Patent Publication 4) JP-A-9-262282
(Patent Publication 5) JP-A-9-276393
(Patent Publication 6) JP-A-5-269198

As another method for immobilizing a protein on the surface of a material, one utilizing a photoreaction has also been disclosed. After a photoreactive active group is generated in a protein to be immobilized, it is applied to the surface of a blood vessel substrate and immobilized by irradiation with ultraviolet rays (ref. Patent Publication 7). This method can be applied to collagen, etc., but in order to apply it to a growth factor, etc. it is necessary to examine the conditions in various ways.
(Patent Publication 7) Published Japanese translation No. 2001-502187 of a PCT application

As methods for imparting a function of promoting endothelialization, the above-mentioned methods have been proposed so far, but it is necessary not only to devise a technique to improve the adhesion of cells but also to immobilize on a blood vessel substrate a material that brings about a cell growth effect. It is considered that immobilizing an angiogenic factor is particularly desirable. An immobilization method that does not affect the activity is further desirable. Representative angiogenic factors include bFGF, VEGF, and HGF, but there have been few attempts to immobilize them on an artificial blood vessel substrate. In particular, as far as the present inventors know there is no case in which HGF has been immobilized. With regard to VEGF, JP-A-10-137334 (Patent Publication 8) discloses a film (polyethylene) on which both VEGF and Fn are immobilized. These proteins are immobilized by graft-polymerizing acrylic acid onto a polyethylene film whose surface has been corona discharge-treated and then covalently bonding the proteins to a carboxyl group activated by carbodiimide. The growth and motility of human umbilical vein endothelial cells (HUVEC) increased on this film. However, an in vivo endothelium regeneration effect has not been examined. Furthermore, Masuda et al. (ref. Non-Patent Publication 10) coated an artificial blood vessel substrate comprising a polyurethane tube with a liquid mixture of VEGF, bFGF, heparin, and gelatin that was made photoreactive by introducing a benzophenone group, although this is a similar method to Patent Publication 6 described above. This was then subjected to UV irradiation to give an artificial blood vessel on which gelatin, etc. was photoimmobilized. When such an artificial blood vessel was grafted to a rat abdominal aorta, it was shown that it was effective for endothelialization. That is, after 4 weeks, for a control blood vessel that had not been subjected to the immobilization 30% thereof was endothelialized, whereas for the immobilized blood vessel 50-60% thereof was endothelialized. As described above, there are methods in which VEGF and bFGF have been immobilized, and both were immobilized by covalent bonding. On the other hand, there are no cases in which HGF has been immobilized.
(Patent Publication 8) JP-A-10-137334
(Non-Patent Publication 10) Jinkouzouki (Artificial Organs) 27, 287-292; 1998

Among angiogenic factors, HGF was initially discovered as a hepatocyte growth factor, but it has subsequently been found to have angiogenic action and has been attracting attention (ref. Non-Patent Publications 11 and 12, Patent Publication 9). In particular, its endothelial growth activity is stronger than that of VEGF, and it is a growth factor specific to endothelium in a blood vessel (ref. Non-Patent Publication 13). VEGF is fast-acting compared with HGF in terms of the angiogenic effect, immediately reacts to ischemia (low oxygen state) to promote the growth of vascular endothelial cells, and also exhibits vascular permeability. Furthermore, it is said that microvessels induced by VEGF do not mature but regress in a short period of time (ref. Non-Patent Publication 14). Moreover, although bFGF has high activity, it exhibits an effect in growing various cells, and there is a possibility of side effects.
(Patent Publication 9) JP-A-6-9691
(Non-Patent Publication 11) Bussolino et al., J. Cell Biol. 119, 629-641; 1992
(Non-Patent Publication 12) Grant et al., Proc. Natl. Acad. Sci. USA, 90, 1937-1941; 1993
(Non-Patent Publication 13) Nakamura et al., Hypertension, 28, 409-413, J. Hypertens. 14, 1067-72; 1996
(Non-Patent Publication 14) Carmeliet, Nature Medicine 10, 1102-1103; 2000

From the above points, the application of HGF to an artificial blood vessel material is expected, but there have been no such disclosure examples. In order to immobilize HGF on an antithrombogenic material, it is necessary to devise some kind of method. For example, ePTFE is strongly water-repellent, and an aqueous solution of a protein is normally repelled. Furthermore, if a solution is dried and solidified thereon, it is easily peeled off, and it is therefore necessary to devise a way of immobilizing a protein on a polymer material such as ePTFE. If covalent bonding is employed, the possibility of losing the activity of the protein is high.

The present inventors have already reported a method for immobilizing a growth factor onto a solid phase stably and without a chemical treatment such as covalent bonding. That is, it is a method for modifying a growth factor so as to be collagen-binding. It is possible to connect a collagen-binding domain site of an Fn molecule and various types of growth factors such as EGF, FGF, BMP, and VEGF (ref. Patent Publications 10 to 13, Non-Patent Publication 15). A fusion protein obtained by such a method can be bound strongly to collagen and, moreover, the activity of a growth factor is similar to that of the natural type. That is, this fusion protein is an excellent method for immobilizing a growth factor on a solid phase coated with collagen. However, this method is limited by a solid phase that is coated with collagen. That is, it is thought that even if an angiogenic factor such as HGF is modified so as to be a collagen-binding type, it cannot be immobilized onto, for example, an artificial blood vessel material that is resistant to adsorption of a protein, such as ePTFE.
(Patent Publication 10) JP-A-2001-190280
(Patent Publication 11) JP-A-2002-58485
(Patent Publication 12) JP-A-2002-60400
(Patent Publication 13) WO02/014505
(Non-Patent Publication 15) Ishikawa et al., J Biochem, 129, 627-633; 2001

### DISCLOSURE OF THE INVENTION

### Problems to be Solved by the Invention

It is an object of the present invention to discover a method for stably retaining on an artificial blood vessel material an endothelial cell growth-promoting agent, for example, HGF, which is an angiogenic factor, without impairing its activity, thereby providing an artificial blood vessel having the function of promoting endothelialization.

### Means for Solving the Problems

As a result of an intensive investigation by the present inventors in order to solve the above-mentioned problems, firstly, a novel method for anchoring collagen onto an artificial blood vessel substrate has been found. That is, it has been found that coating an ePTFE tube with a copolymer of polyurethane and polyamino acid (PAU) is effective in maintaining collagen on the surface of the tube for a long period of time. Secondly, we have succeeded in designing an HGF fusion protein in which collagen-binding activity is imparted to an angiogenic factor, for example, HGF. It has been found that by immobilizing this fusion protein on an artificial blood vessel substrate coated in layers with PAU and collagen, an artificial blood vessel having the function of promoting endothelialization can be realized, and the present invention has thus been accomplished.

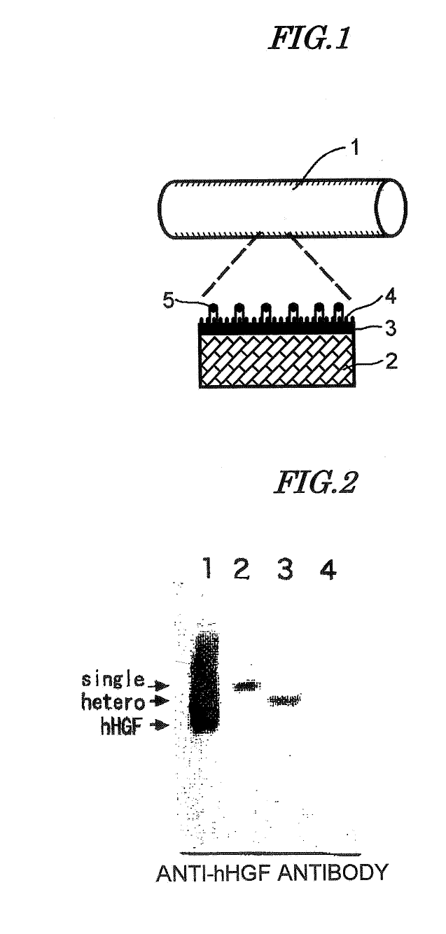

That is, the object of the present invention has been attained by an artificial blood vessel formed by layering and immobilizing in sequence on at least an inner surface of a porous tubular structure (1) a polyamino acid urethane copolymer, (2) collagen or gelatin, and (3) an endothelial cell growth-promoting agent having collagen-binding activity. FIG. 1 shows a schematic view of the internal structure of the artificial blood vessel of the present invention, in which 1 denotes an exterior view of the artificial blood vessel, 2 denotes a porous tubular structure, 3 denotes a polyamino acid urethane copolymer layer, 4 denotes a collagen or gelatin layer, and 5 denotes an endothelial cell growth-promoting agent.

### EFFECTS OF THE INVENTION

The artificial blood vessel of the present invention is prepared by a method that enables a protein to be immobilized onto a substrate without carrying out a chemical treatment such as immobilization by means of covalent bonding, and therefore does not affect the activity of the protein. This preparation method is simple, and since early-stage endothelialization of a grafted artificial blood vessel is possible, an artificial blood vessel that can remain open for a long period of time, even for a small-diameter blood vessel, can be obtained.

### BRIEF DESCRIPTION OF DRAWINGS

(FIG. 1) A schematic view for explaining the structure and function of the artificial blood vessel of the present invention.

(FIG. 2) A diagram showing the result of Western blotting of the HGF fusion protein of the present invention. Lane 1: commercial recombinant HGF protein (natural type). Lanes 2 and 3: culture supernatant protein secreted from AcHH7 cells, 2: prior to serum treatment, 3: serum-treated. Lane 4: culture supernatant of Sf9 cells infected with wild-type virus.

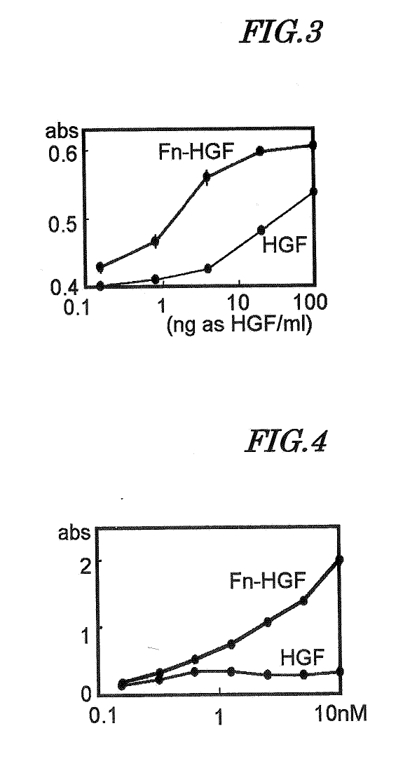

(FIG. 3) A diagram showing a comparison of endothelial cell growth activity of the HGF fusion protein of the present invention with natural HGF.

(FIG. 4) A diagram showing a comparison of collagen-binding activity of the HGF fusion protein of the present invention with natural HGF.

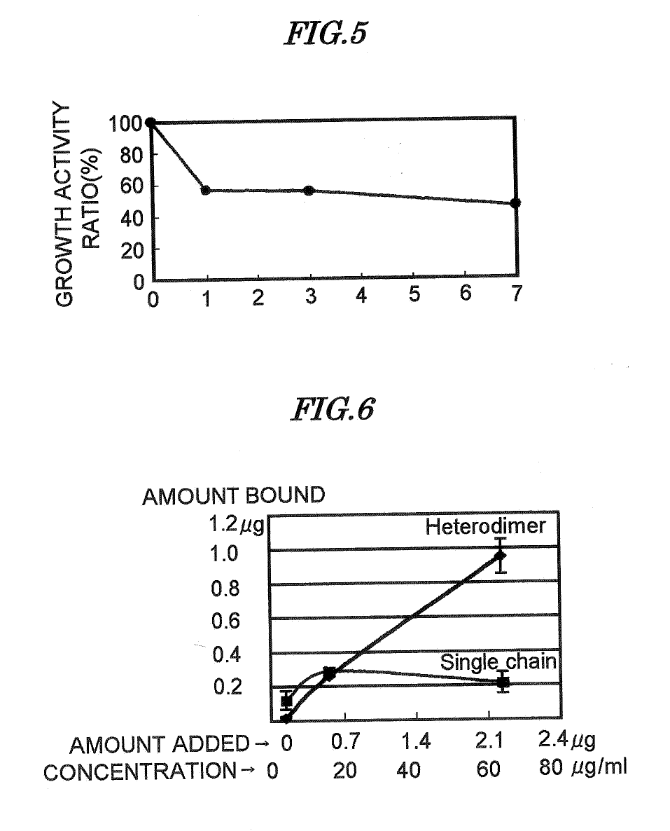

(FIG. 5) A diagram showing changes over time in the activity of the HGF fusion protein of the present invention after binding collagen.

(FIG. 6) A diagram showing the amount of HGF fusion protein bound to a collagen-coated tube.

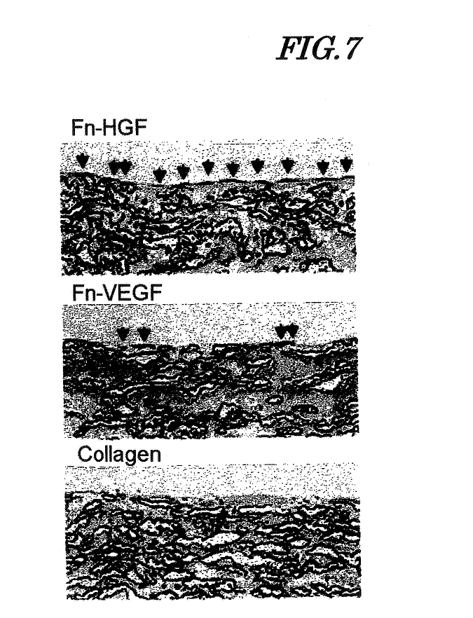

(FIG. 7) A diagram showing that endothelial cells are surviving on the inner surface in the vicinity of a central area of the grafted artificial blood vessel of the present invention.

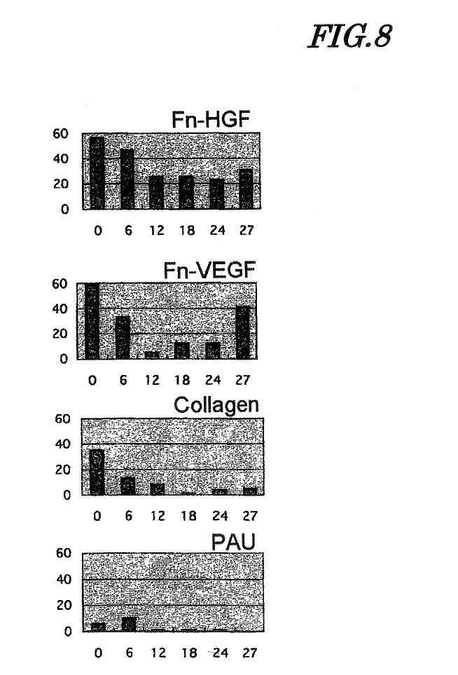

(FIG. 8) A diagram showing the extent of endothelialization along the whole length of the grafted artificial blood vessel of the present invention. The ordinate denotes the number of endothelial cell nuclei present per mm of the periphery, and the abscissa denotes the length (mm) from the anastomosis on the upstream side.

### EXPLANATION OF REFERENCE NUMERALS AND SYMBOLS

1 The artificial blood vessel of the present invention
2 Porous tubular structure
3 Polyamino acid urethane copolymer layer
4 Collagen or gelatin layer
5 Endothelial cell growth-promoting agent

### BEST MODE FOR CARRYING OUT THE INVENTION

The present invention is an artificial blood vessel formed by layering and immobilizing in sequence on at least an inner surface of a porous tubular structure (1) a polyamino acid urethane copolymer, (2) collagen or gelatin, and (3) an endothelial cell growth-promoting agent having collagen-binding activity. As the porous tubular structure, a material and configuration conventionally known as an artificial blood vessel substrate may be used. Examples thereof include a fiber, a woven fabric, and a nonwoven fabric of expanded polytetrafluoroethylene (ePTFE), polyurethane, polyethylene, polyethylene terephthalate (PET), polybutylene terephthalate (PBT), etc., which are porous polymer materials. It may be formed using a porous film-form substrate. As a small-diameter blood vessel substrate, ePTFE and polyurethane are desirable, and in terms of antithrombogenicity ePTFE is particularly desirable.

In the present invention, an inner or outer surface, and at least an inner surface, of a porous tubular structure comprising the above substrate is firstly coated with a polyamino acid urethane copolymer (PAU), which is an amphiphilic polymer having both hydrophobic and hydrophilic regions. PAU may be synthesized by a method disclosed by Uchida et al. (J. Polymer Science, Polymer Chemistry 37, 383-389 (1999; Polymer 41, 473-480 (2000)) or a method published by one of the present inventors (JP-A-2001-136960). A suspension of this polymer in dimethylformamide (DMF) is diluted with dichloroacetic acid to give an appropriate concentration (1 to 3 wt % as a resin concentration), and an ePTFE tube is immersed in the solution thus obtained. Sufficient coating with PAU can be achieved by immersion for on the order of 15 hours, but it can be changed as appropriate depending on the PAU concentration. The tube that has been immersed in PAU is washed with a large amount of distilled water, air-dried, and then heated at 120° C. for 5 minutes to thus dry the PAU. A PAU-coated tube (PAU (+) tube) can thereby be prepared. The amount of PAU coated is suitably in the range of 0.1 to 3 wt % of the porous tubular structure.

The PAU used in the present invention is not particularly limited as long as it has both hydrophobic and hydrophilic regions, but it is preferably a copolymer of urethane and polyamino acid in which an average of 4 or more amino acid units are bonded in sequence, the copolymer being obtained by reacting (a) an α-amino acid-N-carboxylic acid anhydride, (b) a urethane prepolymer having an isocyanate group, and (c) at least one type selected from water, hydrazine, and an organic amine (ref. JP-A-2001-136960).

Next, the PAU (+) tube is coated in a layer with collagen or gelatin. In order to do this, the tube is immersed in, for example, a solution of collagen. If the collagen solution is neutral, it becomes fibrotic, and in order to form a uniform coating it is desirable to use an acidic solution. Alternatively, when a solution having a pH in the vicinity of neutral is used, fibrosis can be suppressed by keeping the solution temperature at 4° C. or less. After immersing in these solutions at 37° C. for 2 hours or at 4° C. for 24 hours, repeated washing with phosphate buffered saline (PBS) is quickly carried out. It is assumed that washing with PBS immobilizes fibrotic collagen on the PAU as a thin layer. Although the amount of collagen or gelatin coated is not particularly limited, it is desirable to immerse the tube in a solution having a collagen or gelatin concentration of 0.001 to 0.5 wt %, and preferably 0.01 to 0.3 wt %. By such a method, the tube is coated with collagen or gelatin at on the order of 0.01 to 5 μg/mm² of the wall surface area.

Stable immobilization of collagen or gelatin can be verified by examining the residual amount from the tube (collagen (+) tube) coated with PAU and collagen in the flow of a liquid. For example, PBS is passed at a rate that is in the range of the blood flow rate in a living body, the tube is left under these conditions for at least 1 week, and the amount of collagen remaining may be subsequently examined by staining using an antibody, etc. When a collagen antibody having good sensitivity cannot be obtained, a method may be employed in which a protein having high binding properties is bound to collagen, and a reaction with an antibody for this protein is carried out. The present inventors have reported that fibronectin collagen-binding domain (FNCBD) can be produced as a recombinant protein (Ishikawa et al., J. Biochem, 129, 627-633; 2001). This FNCBD is bound to the collagen-coated surface as a probe, it is subsequently detected using an anti-FNCBD antibody, and it is thus possible to confirm that collagen remains for a long period of time. As described above, since the PAU-coated surface can maintain a state in which collagen is strongly bound, it becomes possible to further immobilize an endothelial cell growth-promoting agent having collagen-binding activity, for example, an angiogenic factor.

In the present invention, the collagen (+) tube coated in layers with PAU and collagen or gelatin is then coated in a layer with an endothelial cell growth-promoting agent having collagen-binding activity, thus immobilizing it. The endothelial cell growth-promoting agent having collagen-binding activity referred to here means a protein that has both collagen-binding activity and endothelial cell growth-promoting activity. It is preferably an angiogenic factor that has been modified so as to have collagen-binding activity. Examples of the angiogenic factor include HGF, VEGF, bFGF (basic fibroblast growth factor), and EGF (epidermal growth factor), and HGF is particularly desirable. bFGF is not particularly preferable since it exhibits a growth effect for cells other than the blood vessel. With regard to VEGF, it is known that it has a rapid and strong angiogenic effect, but it is a factor that also exhibits vascular permeability, and it might have a problem in terms of blood vessel maturation. However, its use in combination with HGF might be expected to be effective.

As hereinbefore described, in the present invention it is preferable to employ an angiogenic factor that is modified so as to have collagen-binding activity and, in particular, an HGF fusion protein having enhanced collagen-binding activity. Although it has been reported that natural HGF has collagen affinity (Schuppan et al., Gastroenterology 139-152, 1998), the degree thereof is weak, and a majority of the bound HGF is liberated at a physiological salt concentration. Sustained activity as a factor immobilized on an artificial blood vessel can therefore not be expected. In order to exhibit stronger collagen-binding properties it is necessary to design a fusion protein.

The present inventors have proposed a method for imparting strong collagen-binding properties to a growth factor (ref. JP-A-2001-190280, JP-A-2002-58485, JP-A-2002-60400, and WO02/014505), and this method can be applied to HGF. That is, a protein in which a sequence selected from the amino acid sequence of the fibronectin collagen-binding domain (FNCBD) is fused with an amino acid sequence of HGF has been designed. It has been found that good results can be obtained by selecting the above-mentioned FNCBD sequence from, for example, a sequence of amino acid positions 260 to 484 of the mature Fn protein or a sequence of positions 260 to 599. These polypeptides comprising a fibronectin collagen-binding domain may preferably be used in the present invention in order to generate a fusion protein.

A hybrid polypeptide, proposed by the present inventors in JP-A-2002-60400, that is useful as a DDS (drug delivery system) for an angiogenesis regulatory factor maintains angiogenesis regulatory activity by linking the fibronectin collagen-binding domain and the angiogenesis regulatory factor and is a collagen-binding angiogenesis regulatory factor to which binding activity toward collagen has been imparted, and such a polypeptide may be used as one of the endothelial cell growth-promoting agent or the fusion protein of the present invention.

The method previously proposed by the present inventors is a system in which a fusion protein is produced using E. coli. Although an HGF fusion protein could be produced by this system, it exhibited hardly any activity as HGF. It is surmised that when it is produced using E. coli, a complicated tertiary structure cannot be reproduced. In the present invention, the HGF fusion protein employs a method other than that involving E. coli, for example, a baculovirus expression system using an insect cell as a host (Summers and Smith, Texas Agricultural Experiment Station Bulletin No. 1555; 1987). When designing a fusion protein, in order to facilitate secretion from the insect cell, a signal peptide sequence is added to the amino acid terminal. Examples thereof include a signal peptide of a protein secreted from an insect, such as bee venom melittin. This enables a gene coding for a fusion protein having the structure ‘signal peptide-FNCBD-mature HGF protein sequence’ to be designed, and by co-transfecting a DNA having the gene incorporated thereinto and a DNA of the wild-type baculovirus, a recombinant virus is generated within the insect cell. An HGF fusion protein (Fn-HGF) can be translated from a fusion gene coding for the HGF fusion protein incorporated into the recombinant virus, secreted into the culture supernatant of the insect cell, and recovered. Examples of the sequence of this fusion protein are shown by SEQ ID NOS:2 and 4 in the sequence listing.

Therefore, a particularly preferred artificial blood vessel in the present invention is one employing an HGF fusion protein having an amino acid sequence shown by SEQ ID NO:2 or 4 in the sequence listing or an amino acid sequence that is homologous thereto as a fusion protein having collagen-binding activity. In the present invention, the homologous amino acid sequence means an amino acid sequence of a protein having substantially the same collagen-binding activity, in which one or several amino acids are deleted, replaced, inserted, or added.

The HGF fusion protein, preferably used in the present invention, having an amino acid sequence shown by SEQ ID NO:2 or 4 in the sequence listing or an amino acid sequence homologous thereto and having collagen-binding activity may be expressed using a gene comprising a base sequence shown by SEQ ID NO:1 or 3 in the sequence listing or a base sequence homologous thereto. In the present invention, it is preferable to employ a method in which an insect cell is used for such expression of the HGF fusion protein using the gene. The homologous base sequence referred to here means a base sequence coding for an amino acid sequence in which one or several amino acids of the amino acid sequence coded by the corresponding base sequence are deleted, replaced, inserted, or added.

A method for producing a fusion protein is explained in detail below, taking the HGF fusion protein as an example. As a production method, it is also possible to use a method employing not only the above-mentioned baculovirus expression system using an insect cell as a host, but also a mammalian cultured cell, for example, a COS cell. In this case, a large number of appropriate vectors are known, and selection may be made therefrom. As the signal peptide it is possible to use an HGF signal peptide, etc. The HGF fusion protein secreted from the host cell may be purified by putting the culture supernatant on a heparin affinity column. Since this protein has a collagen-binding FNCBD moiety, it may also be purified by a gelatin affinity column, but in order to elute the adsorbed protein from the column it is necessary to use 8 M urea, which causes deactivation, and it is therefore necessary to employ a method for regenerating the tertiary structure. In order to facilitate purification, host insect cells (Sf9, etc.) are cultured in a serum-free culture medium, but in this case the produced HGF fusion protein remains as a single strand. It is said that natural HGF is synthesized as a single strand and then cleaved into α chain and β chain, which are associated to give an active heterodimer (Naka et al., J. Biol. Chem., 267, 20114-20119; 1992). The HGF fusion protein of the present invention can also become a heterodimer by mixing with serum (2 to 10 wt %) and incubating (FIG. 2). It has been confirmed that an HGF fusion protein in the form of a heterodimer has a similar endothelial growth activity to that of natural HGF (FIG. 3), and has strong collagen-binding properties (FIG. 4). Moreover, since the binding properties and the growth activity are stable for a long period of time, it has suitable properties for immobilization onto an artificial blood vessel substrate coated with PAU and collagen or gelatin.

The activity of the HGF fusion protein as HGF may be examined by adding the protein to a culture solution of endothelial cells (HUVEC, HCAEC, etc.). When carrying out this investigation, the activity can be recognized more clearly by comparing with endothelial cells cultured in a culture medium not containing bFGF, VEGF, HGF, etc., which are angiogenic factors. Possession of collagen-binding properties may be ascertained by, for example, adding and binding a solution of the protein to the collagen-coated surface, washing with PBS, etc., then reacting with an anti-HGF antibody, and examining the amount bound. When natural HGF is examined by the same method, its binding saturates at a much lower concentration compared with the HGF fusion protein (FIG. 4). It is therefore difficult to immobilize natural HGF on an artificial blood vessel substrate.

With regard to the stability of binding, after the HGF fusion protein is bound to collagen, it is incubated for a predetermined period of time at, for example, 37° C., and after that the amount thereof bound is measured. This can clarify the retention properties (binding stability) of the HGF fusion protein once it has been bound. Alternatively, after a predetermined period of time has elapsed, cells are seeded, and the retention of activity (stability) may be examined. For example, it has been confirmed that even after 1 week has elapsed substantially 50% of the activity is kept (FIG. 5).

Since the HGF fusion protein of the present invention has the above-mentioned properties, it can easily be immobilized on the surface of a porous tubular structure such as a tube coated in layers in sequence with PAU and collagen, and the binding can be maintained stably. There is the advantage that unlike covalent bonding this method does not affect the molecular structure since it is the Fn collagen-binding domain (FNCBD) that is involved in binding during immobilization. The preparation method merely comprises immersing the above-mentioned coated tube in a solution of a fusion protein. This completes preparation of the artificial blood vessel (HGF-immobilized artificial blood vessel) of the present invention (FIG. 1). Although the amount of endothelial cell growth-promoting agent, for example, HGF fusion protein, coated is not particularly limited, the amount thereof coated is suitably on the order of 2 to 200 ng/mm² of the tube wall surface area.

Actual immobilization of the HGF fusion protein on the artificial blood vessel can be clarified by reacting an antibody for HGF and observing a chromogenic reaction of an enzyme linked to the antibody. When the same detection method is carried out for an artificial blood vessel coated only with collagen and an artificial blood vessel to which natural HGF has been added, the antibody does not bind, and it has been confirmed that there is no coloration. The amount bound may be determined from the difference between the concentration prior to addition of an HGF fusion protein solution and the concentration of a solution recovered after the addition. Alternatively, it may be determined by adding an HGF fusion protein solution to a container with an artificial blood vessel and a container without it, measuring HGF concentrations of the recovered solutions by an ELISA method, and measuring the difference in concentration. It has been confirmed that the amount bound to the substrate coated with PAU and collagen increases depending on the addition concentration up to a concentration of at least 64 μg/mL (FIG. 6).

The effects of the artificial blood vessel (FIG. 1) on which the HGF fusion protein of the present invention has been immobilized can be confirmed by replacement grafting the blood vessel onto an animal blood vessel. An animal having a blood vessel with an internal diameter of about 3 mm is desirable, but since it is said that rat and rabbit have high blood vessel repair ability, canine carotid artery or lower limb femoral artery of a dog is appropriate. By comparing the graft results with, as a control, an ePTFE tube coated only with PAU or only with collagen, it can be confirmed that endothelialization is promoted. As means for confirmation, an artificial blood vessel removed when a predetermined period of time has elapsed after grafting is fixed using formalin, etc., a paraffin section is prepared, and it is stained. It has been confirmed by normal hematoxylin eosin staining that flat endothelial cells are attached to the surface of the artificial blood vessel (FIG. 7). An endothelial layer is formed by such cells being further connected in the form of a sheet. Endothelial cells can be verified by staining with an anti-CD31 antibody, which is a specific antibody, or anti-von Willebrand factor antibody. Alternatively, the removed blood vessel may be subjected to silver staining as it is or fixed using glutaraldehyde, and the surface thereof may be examined using a scanning electron microscope. By the use of these methods, it has been confirmed that endothelial cells are present in substantially the whole area of the artificial blood vessel on which the HGF fusion protein has been immobilized, but with regard to the control artificial blood vessel, it has been confirmed that endothelial cells cannot be observed in a central area, and endothelial cells are present only in the vicinity of the anastomosis of the blood vessel. That is, it is shown that extension of endothelial cells is promoted by the HGF fusion protein (FIG. 8). It is thus possible to clarify that the artificial blood vessel on which the HGF fusion protein has been immobilized is an artificial blood vessel having an endothelialization promotion function. A more specific explanation is given below by way of Examples. In the Examples, % denotes wt % unless otherwise specified.

### EXAMPLE 1

### Production of HGF Fusion Protein (Fn-HGF)

### A) Design of HGF Fusion Protein (Fn-HGF)

### 1) HGF Gene Sequence

As the HGF gene sequence, one disclosed in JP-A-6-9691 was used. This sequence was cloned as a gene coding for a protein exhibiting angiogenic activity, which is produced by a cell line (HUOCA-II and III) established from a human ovarian tumor, and when its base sequence was determined, it was identical to the sequence of HGF reported by Miyazawa et al. (BBRC. 169, 967-973 (1989)). By using this sequence as a template, a sequence coding for mature HGF polypeptide was obtained by a PCR method. The PCR primer had a sequence coding for the enterokinase-recognizing amino acid sequence DDDK (D=aspartic acid, K=lysine) added thereto, and this gave a gene coding for a protein having the enterokinase recognition sequence linked to the amino terminal of the mature HGF sequence. The gene sequence thus obtained was digested by Sal I and BamH I restriction enzymes and linked to pBlueScript II SK(−) (manufactured by Stratagene) cleaved by the same two enzymes, thus giving plasmid pHH2.

### 2) Fusion Gene of Fibronectin and HGF

The cDNA sequence of human fibronectin (Fn) has already been reported (Kornblihtt et al., EMBO J. 4, 1755 (1985), in database Genbank X02761, Swiss P02751). A PCR primer was prepared based on this sequence, and a partial sequence of Fn was amplified. That is, first, human kidney-derived RNA was extracted in accordance with Ishikawa et al. (J. Biochem., 129, 627-633) and transformed into cDNA by reverse transcription, and subsequently the DNA of two types of sequence regions was amplified by PCR. One thereof was the sequence with amino acid positions from 260 to 484 as the mature Fn protein, and the other was the sequence from 260 to 599. The two were cleaved by a restriction enzyme, recovered, and inserted into the above-mentioned plasmid pHH2 so that the Fn sequence was linked to the HGF amino terminal. Plasmids pHH3S and pHH3L were thereby obtained. The former had DNA coding for Fn amino acid positions 260 to 484, and the latter had DNA corresponding to positions 260 to 599.

### 3) Preparation of Transfer Vector

pHH3S was digested by restriction enzymes Mst I and BamH I, and a 2.85 Kb BamH I digested fragment was isolated. This fragment was inserted into the BamH I site of pAcYM1-MeI (Tomita et al., Biochem. J. 312, 847-853 (1995)) to give transfer vector pHH7. In this vector, DNA sequences coding for bee venom protein melittin signal sequence, human Fn sequence (Ala 260 to Arg 484), enterokinase recognition sequence DDDDK (D=aspartic acid, K=lysine), and mature HGF sequence respectively were linked in sequence without the reading frame being displaced. The sequence of this fusion gene is shown in the sequence listing SEQ ID NO:1. The melittin signal sequence was added in order to secrete the fusion protein outside the cell, and it was cleaved out when secreting. The amino acid sequence of the secreted fusion protein, that is, the HGF fusion protein (Fn-HGF), is shown in the sequence listing SEQ ID NO:2.

Using the same procedure as above, a 3.2 Kb BamH I digested fragment isolated from pHH3L was inserted into pAcYM1-MeI to give transfer vector pHH7L. The gene sequence of the fusion protein is shown in the sequence listing SEQ ID NO:3, and the amino acid sequence of the secreted HGF fusion protein is shown in the sequence listing SEQ ID NO:4.

### B) Production and Purification of HGF Fusion Protein (Fn-HGF)

### 1) Preparation of Recombinant Virus

(1) First, 1×10⁶ Sf9 insect cells were suspended in Grace's Medium (Gibco, Invitrogen Corporation) to which 10% fetal calf serum (FCS) had been added, and placed in a culture dish having a diameter of 35 mm. After allowing it to stand for 30 minutes, the culture medium was removed, and the culture dish was washed three times with Sf-900 II serum-free culture medium (Gibco, Invitrogen Corporation).
(2) 2 μg of pHH7 transfer vector DNA was subjected to ethanol precipitation, dried, and then dissolved in 3.5 μL TE (10 mM Tris-HCl (pH 8)/1 mM EDTA). This was mixed with 0.1 μg (1 μL) of Baculovirus linear DNA (Baculogold, Pharmingen) and then with sterile distilled water (DW) to make a total amount of 8 μL. 8 μL of doubly diluted lipofectin (Gibco, Invitrogen Corporation) was added to the mixture to make 16 μL. 15 minutes after mixing, 5 μL (or 11 μL) thereof was added together with 1 mL of Sf-900 II, to the culture dish of (1) above from which the culture medium had been removed.
(3) After culturing was carried out at 28° C. overnight, the liquid was removed, 1 mL of Grace's culture medium (10% added serum) was added, and culturing was carried out for a further 3 days. This culture supernatant was recovered and stored as a virus liquid.
(4) Isolation of recombinant virus

1×10⁶ Sf9 cells were seeded onto a 35 mm φ culture dish and allowed to stand for 30 minutes, and the culture medium was then removed by suction while leaving about 200 μL thereof. The stored culture supernatant (virus liquid) of (3) above was diluted with Grace's culture medium at 100, 1000, and 10000 times and 100 μL thereof was added to the culture dish, thus infecting the cells. The liquid was stirred every 15 minutes, and after this was carried out four times (after 1 hour), the liquid was removed by suction. 3% low-melting agarose treated in advance in an autoclave was diluted with Grace's culture medium (containing 10% serum) at 3 times and incubated at 37° C., and 2 mL thereof was added to the cells in the above-mentioned culture dish from which the virus liquid was removed. After it was allowed to stand at room temperature for 30 minutes and thus solidify, 1 mL of Grace's culture medium (containing 10% serum) was added thereto, and this was cultured at 28° C. for 5 days. After culturing, 1 mL of Neutral red (0.1 mg/mL) was added, and the mixture was allowed to stand for at least 4 hours. This enabled a plaque formed by cells that had lysed in the infected site to be differentiated. Agarose of a single plaque area was punched out by means of a Pasteur pipette and suspended in 500 μL of Grace's culture medium (containing 10% serum) to thus liberate the virus, which was stored at 4° C. The above-mentioned procedure was repeated using the virus liquid thus obtained, and purification was carried out until a single virus clone (AcHH7) was obtained. The infectious virus count (titer) per unit volume of recovered liquid may also be measured by the above-mentioned plaque formation method. When the purified virus was obtained, infection was repeated while gradually increasing the scale of the culture, thus giving a large amount of the virus liquid.

### 2) Confirmation of HGF Fusion Protein Expression

After 1×10⁶ Sf9 cells were seeded onto a 35 mm φ dish, infection with AcHH7 virus at an m.o.i of 5 or 10 was carried out. Culturing was carried out for 4 days after infection, and the culture supernatant was recovered. Secretion of the HGF fusion protein into the culture supernatant from the AcHH7-infected cells was confirmed by an immunoblot (Western blot) technique. That is, first, the culture supernatant was subjected to SDS-polyacrylamide (7.5%) electrophoresis (SDS-PAGE) in accordance with the Laemmli method. In this process, electrophoresis was carried out under non-reducing conditions in which mercaptoethanol was not added to a sample buffer. After the electrophoresis, transcription onto a PVDF membrane was carried out by a semi-dry method using a Tris-glycine buffer solution (current of 2 mA/cm² for 90 minutes). The transcribed PVDF membrane was washed with PBS twice and blocked using 25% Block Ace (Dainippon Pharma Co., Ltd.)/PBS for 60 minutes. After washing once with a washing liquid (0.05% Tween-20/PBS), a reaction with an anti-human HGF antibody (T-7701, Institute of Immunology, diluted with 5% Block Ace at 1:1000) was carried out for 60 minutes. After washing with the washing liquid three times, a reaction with a biotin-labeled anti-mouse antibody (DAKO E0464, diluted at 1:500) was carried out for 30 minutes. After washing with the washing liquid three times, a reaction with POD-labeled streptavidin (DAKOP0397, diluted at 1:700) was carried out for 30 minutes. After washing with the washing liquid three times, when HGF bands were subjected to detection with an ECL Western blot detection reagent (Amersham Bioscience), an antibody reactive band was observed in the culture supernatant. Since this band reacted with an anti-fibronectin antibody, it was confirmed that the HGF fusion protein (Fn-HGF) was secreted into the cell supernatant.

### 3) Large-Scale Culturing

First, culturing of 3-5×10⁶ Sf9 cells/mL was started in an Sf-900 II culture medium containing 10% serum (FCS) in an Erlenmeyer flask (polycarbonate Erlenmeyer flask, vent type, Corning). Culturing was carried out in a rotary incubator at 28° C. at a rotational rate of 120 rpm. The first culture scale was 50 mL of culture liquid in a 250 mL flask for 2-3 days, and subculturing was carried out. After the subculturing, 250 mL of culture liquid was used in a 1000 mL flask. In the process of subculturing this sample, the serum concentration was gradually decreased to 10%, 5%, 2%, and 1%, and culturing was finally carried out in a serum-free culture medium (Sf-900 II). The cells naturalized to the serum-free state were cultured at a cell concentration of 2×10⁶ cells/mL (Sf-900 II, serum-free, rotary culturing). The cells were recovered by centrifugation and infected with recombinant virus AcHH7 at an m.o.i of 5 to 10 (1 hour at room temperature). In this process, stirring was carried out appropriately, the amount of the liquid was adjusted so as to be the amount prior to centrifugation by adding serum-free culture medium, and the liquid was returned to rotary culturing. After culturing was carried out for 3 days, the culture supernatant was recovered. When the supernatant thus recovered was stored, it was cryopreserved at −80° C.

### 4) Purification

(1) CHAPS solution was added to the culture supernatant obtained in the Example above to give a final concentration of 0.03%, and the mixture was filtered with a 0.45 μm filter.
(2) Purification was carried out as follows using FPLC. A heparin column (Hitrap Heparin HP, 1 mL, Amersham Bioscience: 17-0406-01) was equilibrated in advance by passing at least 10 mL of a buffer solution at a flow rate of 0.5 mL/min. The composition of the buffer solution was 10 mM phosphate buffer solution (PB), 0.15 M NaCl, and 0.03% CHAPS (pH 7.2).
(3) The sample solution treated in (1) above was added to the column at a flow rate of 0.5 mL/min. The column was then washed with at least 20 mL of the buffer solution at a flow rate of 0.5 mL/min.
(4) Elution was carried out by passing the buffer solutions below at a flow rate of 0.5 mL/min.
Elution buffer solutions: liquid A 0.15 M NaCl, 10 mM PB, 0.03% CHAPS (pH 7.2); liquid B 2M NaCl, 10 mM PB, 0.03% CHAPS (pH 7.2)

Liquid A and liquid B were mixed so as to give the NaCl concentrations below and used.

Washing with 0.15 M NaCl for 5 minutes.
Eluting with 0.4 M NaCl for 40 minutes.
Eluting with 0.7 M NaCl for 40 minutes.
Fraction size=2 min
Washing with 2 M NaCl for 20 minutes.
(5) The degree of purification of a second peak of the 0.7 M NaCl elution fraction was checked by means of SDS-PAGE.
(6) Fractions for which the presence of Fn-HGF was confirmed were pooled and subjected to dialysis with 10 mM PB, 0.15 M NaCl, and 0.03% CHAPS (pH 7.2) for 24 hours. This was used as a purified sample of the HGF fusion protein. After dispensing, they were stored at −80° C.

### (7) Quantification

The concentration of the purified sample was quantified by the ELISA method using Hymnis HGF ELISA (CODE 1EH1, Institute of Immunology). That is, the sample concentration was expressed as the amount of HGF.

### (8) Heterodimerization

Since the Fn-HGF thus produced was cultured, recovered, and purified under serum-free conditions, it can be expected to be a single strand form. Fetal calf serum (FCS) was added thereto at 10% and the mixture was incubated at 37° C. for 15 minutes. In accordance with the method of (2) above, human HGF (hHGF, natural type) (lane 1), fusion protein prior to serum treatment (lane 2), treated protein (lane 3), and recombinant virus-uninfected Sf9 cell culture supernatant (lane 4) were each subjected to electrophoresis and then Western blotting (FIG. 2). The change in mobility as a result of the serum treatment suggested that the Fn-HGF had been altered into a heterodimer.

### C) HGF Fusion Protein (Fn-HGF) Activity

### 1) Collagen-Binding Activity

First, 100 μL each of a 10 μg/mL PBS solution of type 1 collagen (bovine) (CELLGEN, Koken Co., Ltd.) was pipetted into an ELISA 96 well plate (Nunc Polysorp 96 well immuno module) while carrying out ice-cooling, and incubated at 4° C. for 24 hours. After the well was washed with a washing liquid (0.05% Tween-20/PBS) three times, 250 μL of a 50% Block Ace/PBS solution was pipetted and incubated at room temperature for 60 minutes. After washing three times, 100 μL of various concentrations of Fn-HGF solution were pipetted and incubated at 37° C. for 120 minutes. After washing the wells five times, anti-human HGF antibody (diluted at 1:1000) was incubated for 120 minutes. After washing five times, POD-labeled anti-mouse antibody (DAKO P0260, diluted at 1:1000) was incubated for 60 minutes. After washing five times, 100 μL of a solution of an enzyme substrate (OPD, Sigma Inc.) was pipetted and incubated at room temperature for 30 minutes. 50 μL of 2 N sulfuric acid was added to each well to terminate a reaction, and the absorbance (492 nm-620 nm) was measured.

As shown in FIG. 4, Fn-HGF bound strongly to bovine type 1 atelocollagen with concentration dependency, but HGF (TOYOBO HGF-101, CHO cell expressing recombinant protein) exhibited only a small degree of binding. In the case of HGF, the amount of binding increased, though slightly, up to an addition concentration of 0.5 nM (protein concentration about 50 ng/mL), but no increase in the amount of binding could be seen at concentrations above that. It is surmised that, as reported by Schuppan et al. (Gastroenterology, 114, 139-152 (1998)), such a result is due to collagen having weak binding properties to HGF. According to the results reported therein, the amount of binding of HGF at this concentration was about 1.5 ng, which was about 4% of the amount added. It can be said that binding of HGF is already saturated at this stage. On the other hand, Fn-HGF exhibited higher binding properties than HGF at all concentrations, and the amount of binding increased in response to an increase in the addition concentration. Moreover, it was found that even when the concentration of the solution added was 20 times that in the case of HGF, the binding was not saturated, and stronger collagen-binding properties were imparted. It was found by a similar examination that, as well as type 1 Fn-HGF, type 2, type 3, and type 4 all exhibited high binding activity.

### 2) Cell-Growth Activity for Vascular Endothelial Cells

### (1) Activity of HGF Fusion Protein in Solution

Human coronary artery vascular endothelial cells (ACBRI, Dainippon Pharma Co., Ltd.) were suspended in a culture liquid (base culture medium=EBM-2, Clonetics Inc., added reagent=IGF-I, 2% FCS), and 1×10⁴ cells/500 μL per well were seeded onto a 24 well culture plate (Falcon 24 well plate). After confirming that the cells had adhered, 5 μL of Fn-HGF solution was added thereto, and culturing was carried out at 37° C. under 5% CO₂ at a humidity of 100%. After 3 days, 50 μL/well of WST-1 test liquid (Dojindo Laboratories) was pipetted, and the absorbance (450-620 nm) of each well after 4 hours was measured. From the results, a concentration-dependent vascular endothelial cell growth effect was shown within a range of 0 to 100 ng/mL expressed as an HGF concentration. Moreover, the resulting value exceeded the activity exhibited by HGF on its own. It is therefore surmised that, in the HGF fusion protein of the present invention, designing it as a fusion protein not only enables the original growth activity to be maintained but also gives the possibility of achieving a stable effect (FIG. 3).

(2) Cell-Growth Activity after Binding Collagen

500 μL of a 10 μg/mL PBS solution of bovine type 1 collagen (ice-cooled) was pipetted into a 24 well culture plate and incubated at 4° C. for 24 hours. After washing with PBS five times, 500 μL of Fn-HGF solution was pipetted and incubated at 37° C. for 2 hours. After the plate was washed with PBS five times, it was stored at 37° C. in PBS. Immediately after storing, 1 day thereafter, 3 days thereafter, and 7 days thereafter, 1×10⁴ cells/500 μL of human coronary artery vascular endothelial cells (HCAEC) were seeded onto different wells respectively using a culturing liquid (base culture medium=EBM-2, added reagent=IGF-I, 2% FCS, Clonetics). From this point on, the cell-growth activity was examined by the WST-1 method in the same manner as in (1) above. From the results, the activity of Fn-HGF decreased by about 60% after 1 day, but after that the activity was maintained stably up to 1 week (FIG. 5).

### EXAMPLE 2

### Production of PAU

PAU was synthesized in accordance with a method described in Example 1 of JP-A-2001-136960. That is, 980 g of polytetramethylene ether glycol (OH value 57.35) and 174 g of tolylene diisocyanate (a mixture of 2,4-tolylene diisocyanate and 2,6-tolylene diisocyanate, 2,4-tolylene diisocyanate 80 wt %) were reacted at 70° C. for 5 hours, thus giving a urethane prepolymer having a terminal isocyanate group (NCO equivalent 1165). 58.2 g of the urethane prepolymer and 58.2 g of γ-methyl-L-glutamate-N-carboxylic acid anhydride were dissolved in 394.3 g of dimethylformamide (DMF), and a solution formed by dissolving 1.375 g of hydrazine hydrate in 20 g of DMF was added dropwise thereto to effect a reaction, thus giving a polyamino acid urethane copolymer (PAU) solution (20% concentration DMF solution) having a viscosity of 18500 cp/25° C. The average degree of polymerization of amino acid chains was about 62 when calculated based on the reactivity between a primary amine and an isocyanate and the mechanism of polymerization of an N-carboxylic acid anhydride by a primary amine (Murray Goodman and John Hutchison, J. Am. Chem. Soc., 88, 3627 (1966)).

### (Preparation of PAU-Coated Artificial Blood Vessel)

Coating of an artificial blood vessel substrate was carried out as follows, using the PAU obtained above. First, the PAU solution was diluted with dichloroacetic acid to give a PAU concentration of 2%. An artificial blood vessel ePTFE tube (internal diameter 3 mm, length about 35 mm) was soaked in ethanol in advance so as to remove bubbles and immediately immersed in the above-mentioned PAU solution. After allowing it to stand at 4° C. for 15 hours, it was put into 500 mL of distilled water, the distilled water was replaced three times, and it was then kept in distilled water at room temperature for 24 hours (constant stirring). Following this, it was washed with 500 mL of distilled water three times. The tube thus washed was air-dried, then moved to an oven at 120° C., and heated for 5 minutes, thus giving a PAU-coated tube (PAU (+) tube). From the difference in weight between that before coating and that after coating, it was found that the tube was coated with PAU at a weight corresponding to 0.5% of the weight of the ePTFE.

### (Preparation of Collagen-Coated Artificial Blood Vessel)

After the above-mentioned PAU (+) tube was washed with methanol, and then with 70% methanol three times, it was washed with distilled water three times. Subsequently, it was placed in a 0.2% collagen acidic solution (1-PC collagen 0.5% diluted with distilled water, Koken Co., Ltd.) and treated at 4° C. for 15 hours. After the treatment, it was washed with PBS three times, thus giving a collagen-coated tube (collagen (+) tube) in which the PAU was coated in a layer with collagen.

### (Confirmation of Collagen Coating)

After the collagen (+) tube was washed with PBS, it was soaked in a 40 μg/mL (1 μM) FNCBD solution.

The FNCBD was produced in accordance with a method described in JP-A-2001-190280. After carrying out a reaction at 37° C. for 2 hours, it was washed with PBS three times. The tube thus washed was inserted into a 4 cm long polypropylene tube and placed in an environment in which PBS was flowing. This tube had a gradually narrowing shape in which the upstream end had an internal diameter of 5 mm and the downstream end had an internal diameter of 2.5 mm. This prevented the inserted ePTFE tube from being carried along by the PBS. This polypropylene tube was connected to a silicone tube set in a rotary pump, and PBS was circulated at a flow rate of 100 mL/min. 4 collagen (+) tubes were prepared, each was placed in the PBS circulation path for a predetermined period of time, and all were taken out after 12 days. That is, collagen (+) tubes exposed in the flow path for 1, 2, 6, and 12 days were recovered. They were simultaneously subjected to examination of stainability by an anti-FNCBD antibody. An anti-FNCBD monoclonal antibody (FC4-4, TaKaRa Bio Inc.) was diluted at 1:1000, and a reaction was carried out at room temperature for 1 hour. Subsequently, they were washed with PBS three times, and bonded antibody was detected by an ABC method (Vectorstain, mouse ABC-PO kit, AB Vector LLC). That is, biotinylated anti-mouse IgG antibody and ABC-complex were reacted in sequence in accordance with a manual of AB Vector LLC. After the reaction, washing was carried out with PBS six times, and a staining reaction was then carried out using chloronaphthol/H₂O₂. From the results, it was confirmed that after coating with collagen, the collagen remained for up to 12 days with hardly any change. This suggests that PAU is a very effective material for collagen to adhere to.

(Preparation of Artificial Blood Vessel with HGF Fusion Protein Immobilized Thereon)

First, in order to examine what level of HGF fusion protein could be bound to the collagen (+) tube, the following examination was carried out. A plurality of 4 mm diameter disks punched out of the collagen (+) tube were prepared. Various concentrations of HGF fusion protein solution and PBS were added to wells of a 96 well plate. 4 wells each were prepared at the same concentration, the above-mentioned disks were placed into two thereof, and no disk was placed in the remaining two wells. After keeping them at 37° C. for 2 hours, the solutions were recovered from the wells, and their HGF concentrations were measured using an HGF ELISA kit (Quantikine HGF, R&D Co., Ltd.). The concentration of HGF fusion protein bound was determined by subtracting the HGF concentration of the wells in which the disk was placed from the HGF concentration of the wells in which no disk was placed. The results are given in FIG. 6. As is clear from FIG. 6, when the HGF fusion protein was made into a heterodimer, the amount of binding increased according to the amount added. At a maximum concentration of 64 μg/mL, about a half of the amount added was bound. On the other hand, in the case of a single strand molecule, binding was saturated at a low concentration.

Next, HGF fusion protein solutions with a possible maximum addition concentration of 64 μg/mL (as HGF concentration) and 16 μg/mL were added to the above-mentioned collagen (+) tubes. When the amount of HGF fusion protein bound was measured in the same manner as above, the results below were obtained (tested with 3 tubes each and 1 mL each of liquid). That is, the amount bound and the immobilized HGF density were 11.7 μg and 21 ng/mm² per tube respectively in the case of 64 μg/mL, and 4.5 μg and 8 ng/mm² per tube respectively in the case of 16 μg/mL.

### EXAMPLE 3

### Grafting onto HGF Fusion Protein-Immobilized Artificial Blood Vessel

The artificial blood vessel, prepared in Example 2, onto which HGF fusion protein was immobilized (addition concentration 64 μg/mL), was replacement grafted to a lower limb femoral artery of a beagle dog. After a 3 cm long artery was removed, a 3 cm long artificial blood vessel was suture-grafted. 1, 2, and 4 weeks after grafting the artificial blood vessels were removed, formalin-fixed, and embedded in paraffin. The paraffin block was equally divided into 10 parts along the longitudinal axis of the artificial blood vessel, and a paraffin section was prepared from each part and stained with hematoxylin and eosin; from the results, no endothelial cells were observed on the inner surface of the artificial blood vessel after 1 week. For the sample after 2 weeks, endothelial cells were observed in a site up to about 3 mm from the anastomosis (upstream side) with the living blood vessel, but no endothelial cells were observed on the downstream side.

On the other hand, for the sample removed after 4 weeks, endothelium-like cells were observed over the whole area. That is, nuclei of endothelium-like cells were observed on the inner face of the artificial blood vessel for all 10 parts. FIG. 7 shows an image of a section in the vicinity of the central area of the blood vessel (site furthest from the two ends). When these endothelium-like cells were stained with an anti-CD31 antibody, they gave a positive result. Furthermore, the number of nuclei of endothelial cells on the inner surface was counted for 6 parts out of the 10 parts. FIG. 8 shows the count as the number of endothelial cells present per mm of the peripheral length. As is clear from these results, the Fn-HGF-immobilized artificial blood vessel was endothelialized over the whole length.

### EXAMPLE 4

### Grafting of VEGF-Immobilized Artificial Blood Vessel

Collagen-binding VEGF was added and immobilized on the collagen (+) tube prepared in Example 2. The addition concentration was 0.8 μM (80 μg/M) as for the HGF fusion protein. This collagen-binding VEGF was a VEGF fusion protein already disclosed by the present inventors (ref. JP-A-2002-60400).

The VEGF-immobilized blood vessel thus prepared was grafted to a lower limb femoral artery of a beagle dog in the same manner as in Example 3 above.

### COMPARATIVE EXAMPLE 1

### Grafting of PAU-Coated Artificial Blood Vessel

The PAU (+) tube prepared in Example 2 was grafted to a lower limb femoral artery of a beagle dog in the same manner as in Example 3 above.

### COMPARATIVE EXAMPLE 2

### Grafting of Collagen-Coated Artificial Blood Vessel

The collagen (+) tube prepared in Example 2 was grafted to a lower limb femoral artery of a beagle dog in the same manner as in Example 3 above.

All of the tubes grafted in Example 4 and Comparative Examples 1 and 2 were removed after 4 weeks, and paraffin sections were prepared and stained in the same manner as in the case of the HGF fusion protein-immobilized artificial blood vessel of Example 3 above. As is clear from FIG. 8, there were fewer endothelial cells for the VEGF-immobilized artificial blood vessel than for the HGF-immobilized artificial blood vessel, but endothelial cells were observed to be present over the whole length. On the other hand, for the artificial blood vessel with only the PAU coating, a very small number of endothelial cells were observed. In particular, an area from the central part to the downstream end was in a state in which hardly any endothelial cells were present. Furthermore, endothelial cells were present over the whole area of the collagen-coated artificial blood vessel, but the number thereof was much smaller than that of the HGF-immobilized artificial blood vessel. From these results, it is surmised that the immobilization of HGF or VEGF promoted the endothelialization of the artificial blood vessel. This effect was shown more by HGF than by VEGF.

### INDUSTRIAL APPLICABILITY

The artificial blood vessel of the present invention is prepared by a simple method involving layering and immobilization, without affecting protein activity. Since the artificial blood vessel when grafted can undergo early stage endothelialization, an artificial blood vessel that enables even a small-diameter blood vessel to have a long-term patency can be obtained. The artificial blood vessel of the present invention is expected to be used as an artificial blood vessel that can be applied to a site where small-diameter blood vessels are present, such as a coronary artery or a lower limb vessel (below-knee artery).

### (Sequence Listing Free Text)

### SEQ ID NO:1

Explanation of artificial sequence: DNA sequence coding for HGF fusion protein.
Base Nos. 1 to 62; base sequence (derived from pAcYM1-MeI) coding for melittin signal sequence.
Base Nos. 70 to 744; sequence coding for Fn amino acid positions 260 to 484.
Base Nos. 748 to 762; sequence coding for enterokinase recognition sequence.
Base Nos. 763 to 2856; sequence coding for mature HGF polypeptide.

### SEQ ID NO:2

Explanation of artificial sequence: HGF fusion protein
Amino acid positions 3 to 227; amino acid sequence of Fn amino acid positions 260 to 484.
Amino acid positions 229 to 233; enterokinase recognition sequence.
Amino acid positions 234 to 930; amino acid sequence of mature HGF polypeptide.

### SEQ ID NO:3

Explanation of artificial sequence: DNA sequence coding for HGF fusion protein.
Base Nos. 1 to 62; base sequence (derived from pAcYM1-MeI) coding for melittin signal sequence.
Base Nos. 70 to 1089; sequence coding for Fn amino acid positions 260 to 599.
Base Nos. 1093 to 1107; sequence coding for enterokinase recognition sequence.
Base Nos. 1108 to 3201; sequence coding for mature HGF polypeptide.

### SEQ ID NO:4

Explanation of artificial sequence: HGF fusion protein
Amino acid positions 3 to 342; amino acid sequence coding for Fn amino acid positions 260 to 599.
Amino acid positions 344 to 348; enterokinase recognition sequence.
Amino acid positions 349 to 1045; mature HGF polypeptide amino acid sequence.

1. An artificial blood vessel comprising a porous tubular structure and, layered and immobilized in sequence onto at least the inner surface thereof, (1) a polyamino acid urethane copolymer, (2) collagen or gelatin, and (3) an endothelial cell growth-promoting agent having collagen-binding activity.

2. The artificial blood vessel according to claim 1, wherein the porous tubular structure is formed from expanded polytetrafluoroethylene.

3. The artificial blood vessel according to claim 1, wherein the polyamino acid urethane copolymer is a copolymer of a urethane and a polyamino acid having an average of at least 4 amino acid units connected in series, the copolymer being obtained by reacting (a) an α-amino acid-N-carboxylic acid anhydride, (b) a urethane prepolymer having an isocyanate group, and (c) at least one type selected from water, hydrazine, and an organic amine.

4. The artificial blood vessel according to claim 1, wherein the endothelial cell growth-promoting agent is a fusion protein of an angiogenic factor and a polypeptide having collagen-binding activity.

5. The artificial blood vessel according to claim 4, wherein the angiogenic factor is HGF.

6. The artificial blood vessel according to claim 4, wherein the polypeptide having collagen-binding activity is a fibronectin-derived polypeptide.

7. The artificial blood vessel according to claim 4, wherein the fusion protein is an HGF fusion protein having an amino acid sequence shown in SEQ ID NO:2 or 4 in the sequence listing or an amino acid sequence homologous thereto.
